(19) United States
(12) Patent Application Publication
(10) Pub. No.: US 2021/0129136 A1
(43) Pub. Date: May 6, 2021

(54) LATERAL FLOW ASSAY AND DEVICE FOR SKIN CARE APPLICATION

(71) Applicant: FibroTx OÜ, Tallinn (EE)

(72) Inventors: Toomas Neuman, Tallinn (EE); Petrus Johannes Louis Spee, Tallinn (EE)

(21) Appl. No.: 16/635,826

(22) PCT Filed: Aug. 3, 2018
(86) PCT No.: PCT/EP2018/071179

§ 371 Date: Jan. 31, 2020

(30) Foreign Application Priority Data
Aug. 3, 2017 (EP) 17184824.5
Sep. 7, 2017 (EP) 17189907.3

(51) Int. Cl. B01L 3/00; G01N 33/558

(52) U.S. Cl. CPC B01L 3/5023; B01L 3/5029; G01N 33/558; B01L 2200/025; B01L 2300/0887; B01L 2200/12; B01L 2300/043; B01L 2300/0825; B01L 2200/027

(57) ABSTRACT

The present invention relates to diagnostic kits and methods based on lateral flow assay devices for detecting the presence or quantity of one or more test analytes within a test sample taken from the skin of a mammal.

### FIELD OF THE INVENTION

The present invention relates to diagnostic kits and methods based on lateral flow assay devices for detecting the presence or quantity of one or more test analytes within a test sample taken from the skin of a mammal.

### BACKGROUND OF THE INVENTION

Fast development of genomics, transcriptomics, proteomics and regulomics has made it possible to analyze molecular and cellular mechanisms at large scale. One of the important results of these studies has been development of functional genomics and the understanding that cells from different individuals have significant differences in genome structure, gene and protein expression profiles and regulatory mechanisms that control specific cellular functions. This has resulted in an interest in detecting and/or quantifying biomarkers to assess the current state of a mammal by way of presence, absence and/or concentration of one or more biomarkers.

Also there is a need for evaluating how effective treatments are on a personal level, such as in the fields of personalized medicine and personalized skin care.

In relation to personalized skin care the claimed effects of anti-wrinkle and anti-aging effects of cosmetic products are typically based on the assumption that these products have similar effect on all individuals. However, this is not the case. Different people and different skin types react differently to cosmetic products, hence the need for point-of-care devices that can determine the effects or responsiveness of an individual to a particular type of skin care product.

Skin “quality” depends on the biological processes that control and regulate skin morphology, structure and function. Basic biological mechanisms that are responsible for skin performance are related to maintenance, renewal and function of different cell populations in the skin. For example dermal fibroblasts control homeostasis of extracellular matrix, keratinocytes control barrier function of the skin, immune cells and factors are responsible for inflammatory processes and fighting with infections. Functional networks (molecular mechanisms) that control these processes are relatively well known and key players in these networks have been identified. Levels and activity of different cytokines and growth factors regulate balance of cellular processes such as proliferation and differentiation of different cell populations, synthesis and degradation of extracellular matrix, metabolic activity etc. in the skin.

Combination of these activities results in the skin “quality” and aesthetic look of the skin.

Levels of interleukins may be used to determine the status of the skin and also provide recommendations how to improve skin “quality” (appearance, function, structure).

One of the challenges faced with lateral flow assay methods are the provision of a sample to test, in particular the provision of a sample form on the skin, and in particular to provide samples from on the skin in a reproducible and/or uniform manner.

WO 2014184151 A1 describes a point-of-care diagnostic device that is based on lateral flow assay technology and enables non-invasive analysis of secreted and diffusible factors from the skin surface.

US 2005/0175992 describes a method for the rapid diagnosis of targets in human body fluids. In particular a lateral flow assay method is employed, where a sample is collected non-invasively from eye fluid using a swab member.

Consequently there is a need in the art for kits and methods for obtaining and analysing analytes from the skin, in particular point-of-care devices that allows for rapid detection. There is also a need in the art for sampling methods for point-of-care devices that can provide a sample in a reproducible and/or uniform manner compared to the prior art.

### SUMMARY OF THE INVENTION

The present invention was made in view of the prior art described above, and the object of the present invention is to provide a diagnostic kit for detecting the presence or quantity of one or more test analytes within a test sample taken from a skin surface of a mammal.

One aspect of the present invention provides a diagnostic kit for detecting the presence or quantity of one or more test analytes within a test sample obtained from a skin surface of a mammal, the diagnostic kit comprising:
a) a separate swab (200, 301) configured to be used for collecting said test sample, wherein said swab comprising a sample collection pad (201, 101) attached to a supporting member (202),
b) a lateral flow assay device (300) configured to accept and hold said separate swab.

The inventors further provide a modified lateral flow assay to analyze e.g. IL-1a, IL-1RA and IL-8 levels in the skin. Accordingly, a second aspect of the present invention provides a method for detecting the presence or quantity of one or more test analytes, the method comprising the following steps:
a) provide a separate swab (200) comprising a sample collection pad (201) as defined herein, wherein said sample pad comprises a test sample obtained from the skin surface of a subject using said separate swab;
b) insert said swab comprising a sample collection pad containing said test sample in the lateral flow assay device adapted to receive said separate swab insert as defined in any of the preceding claims;
c) developing the lateral flow assay.

### BRIEF DESCRIPTION OF THE DRAWINGS

FIG. 1 shows perspective views of different embodiments, of the present invention, of a porous support assembly (100) (also referred to as a lateral flow assay strip). In FIG. 1a a porous support assembly (100) is shown with a sample pad (101), an conjugate pad (102), a detection zone (105) and an indicator zone (106), both zones immobilized on porous support (107), a wicking pad (104) and a backing material (108). “L” shows the direction of the lateral flow and the area “DA” defines the detection area. FIG. 1b illustrates the porous support assembly, where the sample pad (101) is detached from the remaining porous support assembly. FIG. 1c shows an alternative embodiment of FIG. 1a, where the sample pad (101); conjugate pad (102); detection zone (105) and indicator zone (106) on porous support (107); and wicking pad (104) is adjoining or overlapping, and placed on a backing material (108). FIG. 1d illustrates the lateral flow strip of FIG. 1c, where the sample pad (101) is detached from the remaining porous support assembly.

FIG. 2 shows in an embodiment of the present invention different views of a separate swab (200) comprising a supporting member (202) with an aperture (205) at the distal end (204) of the supporting member (202). The separate swab (200) is disclosed without and with the sample collection pad (201) attached and covering the periphery of the aperture. The supporting member (202) comprises an incision (206) on one of the edges of the supporting member (202). The incision (206) is configured to interact with the bulge (303) on the lateral flow device (300) to orientate and secure the position of the swab in the inserted position in the lateral flow device (300). FIG. 2 further discloses an embodiment of the separate swab (200), where the width of the proximal end (203) of the supporting member (202) is extended to form a finger grip.

FIG. 3 shows in an embodiment of the present invention different views of the lateral flow device (300) with the of the separate swab (200) inserted in a sample pad slot (306), which slot comprises a bulge (303) configured to interact with the incision (206) of the separate swab (200) to orientate and position the separate swab (200) in the lateral flow device (300). The lateral flow device (300) comprises a holding member (302), which may be closed down over the separate swab (200) and hold and lock the swab in the inserted position in the lateral flow device (300). The holding member (302) comprises an opening (304), which in the locked position of the holding member aligns with the sample collection pad (201) such that the sample collection pad is exposed and accessible to running buffer introduced through the opening (304). The lateral flow device (300) further comprises a reaction window (305), which allows visual inspection of the detection area (DA).

FIG. 4 shows the results of in vitro testing different materials for sample collection. 80 μl of a standard protein solution (PBS containing 2 ng/ml IL8, 4 ng/ml IL1A and 8 ng/ml IL1RA recombinant proteins) was used as test sample. Signal intensities are measured as mV.

FIG. 5 shows the results of in vivo testing selected materials for sample collection. FIG. 5A (Forehead) and FIG. 5B (Inner arm). Signal intensities are measured as mV.

FIG. 6 shows the results of comparing blocked sample pads (C083) and the corresponding unblocked sample pad on inner side of a forearm skin. Signal intensities are measured as mV.

FIG. 7 shows the results of comparing sample collection procedures (in vivo). FIG. 7A (forehead) and FIG. 7B (cheek). Three volunteers (JA, AL, and AS). Signal intensities are measured as mV.

FIG. 8 shows the results of comparing different swabbing procedures in vivo on two volunteers (JA and AL). Collection by a Z-shape motion versus 5 seconds within an area of 5 cm2. Signal intensities are measured as mV.

FIG. 9 shows the effect of pre-treatment of the skin prior to sample collection and how long the pre-treatment sustains. FIG. 9A (water pre-treatment of skin), FIG. 9B (EtOH pre-treatment of skin) and FIG. 9C (pre-treatment of skin using a cosmetic wipe). Light grey bar (IL1A). Dark grey bar (IL1RA). Signal intensities are measured as mV.

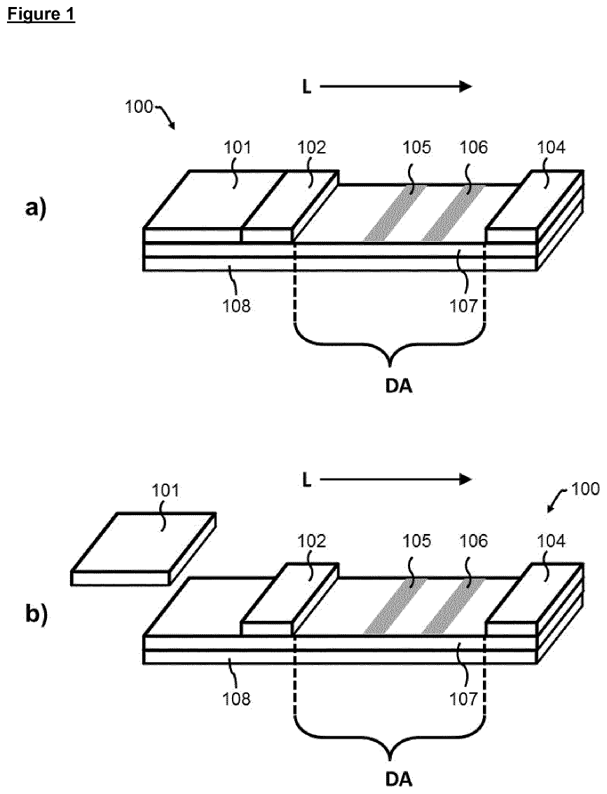

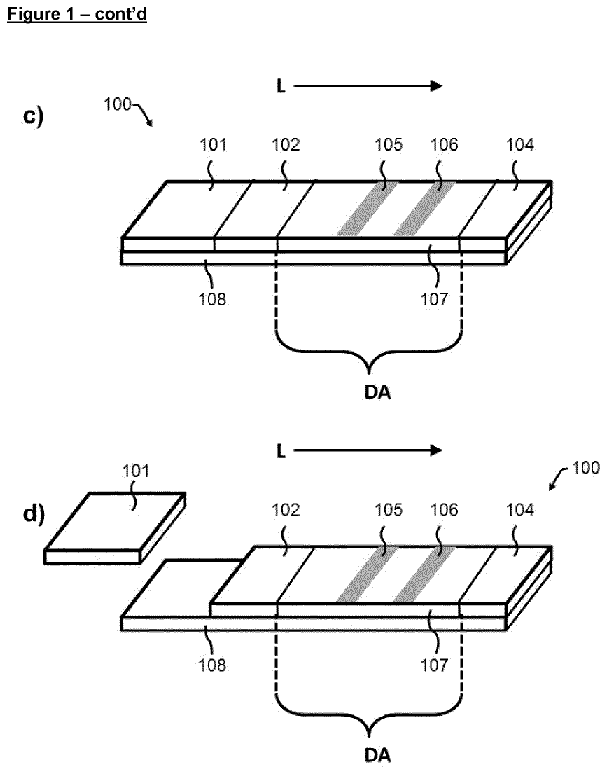

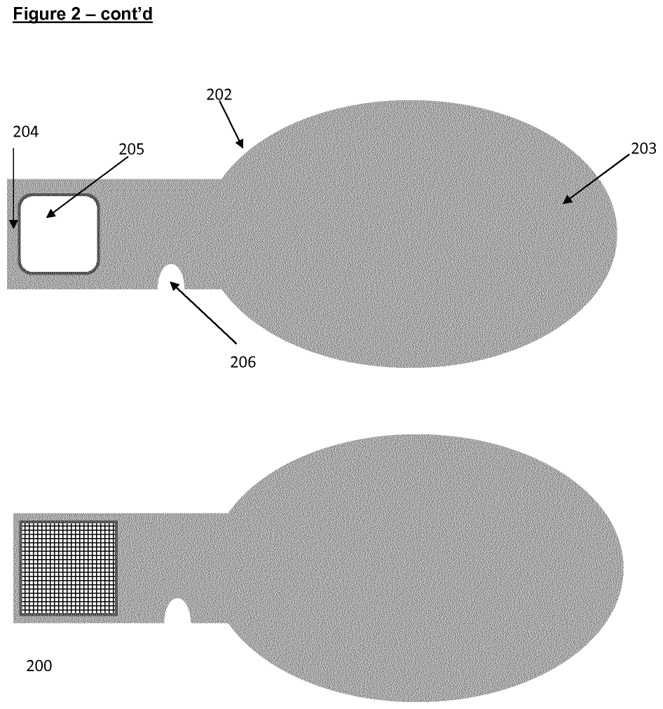

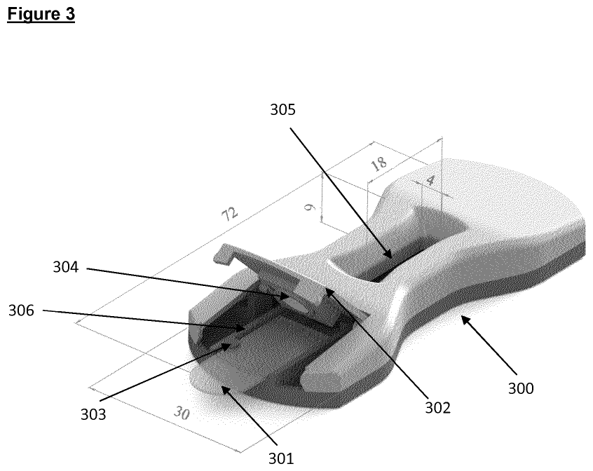

It will be recognized by the person of ordinary skill in the art, given the benefit of this disclosure that certain features shown in FIGS. 1-5 are not necessarily drawn to scale. The dimensions and characteristics of some features in the figures may have been enlarged, distorted or altered relative to other features in the figures to facilitate a better understanding of the illustrative examples disclosed herein.

It will further be recognized by the person of ordinary skill in the art that the individual features of the figures may be interchanged to obtain further embodiments.

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

A first aspect of the present invention provides a diagnostic kit for detecting the presence or quantity of one or more test analytes within a test sample taken from a skin surface of a mammal, the diagnostic kit comprising:
a) a separate swab (200, 301) configured to be used for collecting said test sample, wherein said swab comprising a sample collection pad (201, 101) attached to a supporting member (202),
b) a lateral flow assay device (300) configured to accept and hold said separate swab.

The diagnostic kit of the present invention may be employed in point-of-care devices to detect the presence or absence of one or more test analytes within a test sample obtained from the skin using the separate swab (200) of the diagnostic kit.

The separate swab of the diagnostic kit of the present invention is configured to be suitable for collecting a test sample from the skin surface of a mammal. In a preferred embodiment, the mammal is a human being. The swab (200, 301) comprises a supporting member (202) to which a sample collection pad (201, 101) is attached, preferably on one side of the supporting member. The supporting member (202) is typically used as a handle when the sample is collected from the skin, e.g. by placing the sample collection pad (201, 101) on the skin and moving the pad around on the skin using the supporting member (202) to control the movement.

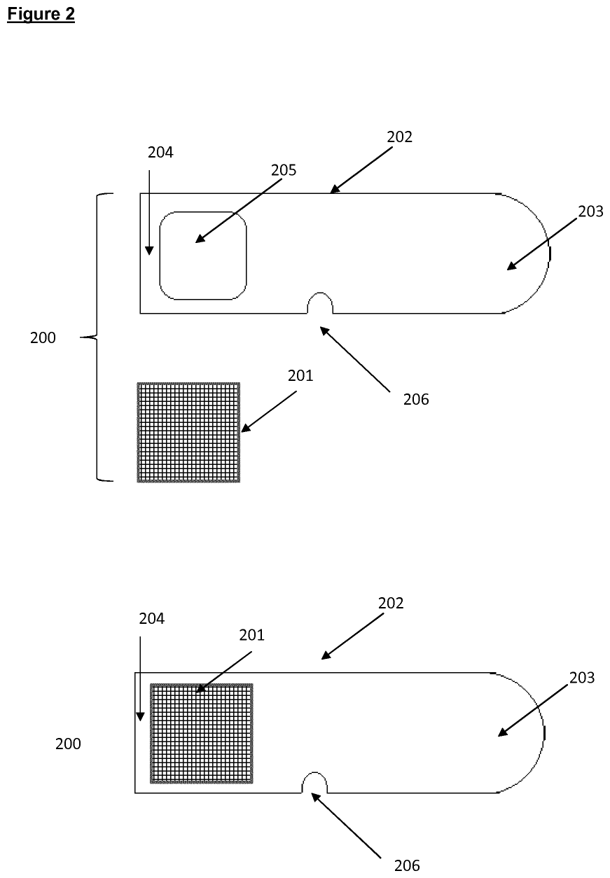

In one embodiment, the supporting member is elongated, for example the length of the member is at least 2 times the width of the member, such as 2.5 times the width of the member, such as 3 times the width of the member, such as at least 4 times the width of the member. In one embodiment, the supporting member is configured with one proximal end (203) configured as a finger grip and opposite distal end (204) to which said sample collection pad (201, 101) is attached. The shape of the proximal end (203) may be configured to allow a firm grip of the supporting member (202) between two or more finger. FIG. 2 discloses an example, where the width of the proximal end (203) of the supporting member (202) is extended to provide a better finger grip. Thus in one embodiment, proximal end (203) of the supporting member is wider than the distal end (204). In one embodiment, the area and shape of the proximal end (203) of the supporting member corresponds to the pulp of an thumb of an adult human being, which allows a firm grip of the supporting member.

In one embodiment, the supporting member (202) is flexible along the longitudinal axis of supporting member. The supporting member (202) may be made of a material that is flexible material such that the supporting member (202) will bend slightly when the sample collection pad (201, 101) is pressed against the skin and moved around on the skin to collect the sample material. The flexibility of the supporting member (202) reduces the risk of injuring the skin. In a preferred embodiment, the supporting member (202) is made of a plastic material, for example the supporting member (202) may be made of a plastic material, where the thickness of the plastic material is less than about 2 mm, such as 1 mm or less, such as between 2 and 0.5 mm, such as between 2 and 1 mm, which makes the supporting member (202) to flexible along the longitudinal axis.

In a preferred embodiment of the present invention, the distal (204) end of the supporting member (202) comprises an aperture (205) configured to be covered by the sample collection pad (201, 101). In this embodiment, the sample collection pad (201, 101) attached to the supporting member (202) covers aperture and the perimeter of the same. In one embodiment, said sample collection pad (201, 101) is attached to the supporting member (202) such that said sample collection pad covers said aperture (205).

The sample collection pad (201, 101) may be attached to the supporting member (202) close to the perimeter of the aperture. The sample collection pad (201, 101) may be attached to the supporting member (202) further away from the perimeter of the aperture. The sample collection pad (201, 101) is typically attached to one side of the supporting member (202). In one embodiment, the area of the aperture (205) corresponds to at least 50% of the area of the sample collection pad (201, 101), such as at least 60% of the area of the sample collection pad, for example at least 70% of the sample collection pad, such as at least 70% of the sample collection pad, for example at least 80% of the sample collection pad, such as at least 90% of the sample collection pad, for example at least 95% of the sample collection pad.

Inserted in the lateral flow device (300), the sample collection pad (201, 101) of the swab (200) forms part of the porous support assembly (100), i.e. the sample collection pad is in contact with the other elements of the assembly. The aperture allows for access to the sample collection pad (201, 101) for the addition of a running buffer to facilitate the lateral flow in the porous support assembly. In the context of the present invention a running buffer is any liquid buffer suitable for facilitate the lateral flow in the porous support assembly, such as a PBS buffer.

The sample collection pad (201, 101) is made of a material that is suitable for collecting the test sample on the skin and subsequent be mated with and form part of the porous support assembly (100) and release the test sample to the porous support assembly (100). In one embodiment, the sample collection pad (201, 101) is made of a cellulose material, a cellulose derivative such as nitrocellulose, polyether sulfone, polyethylene, nylon polyvinylidene fluoride (PVDF), polyester, polypropylene, glass fibers, cotton, or cloth. In a preferred embodiment, the sample collection pad (201, 101) is made of a cellulose material, a cellulose derivative such as nitrocellulose.

The sample collection pad (201, 101) may be in the form of a sheet or the like. In one embodiment, the sample collection pad (201, 101) is in the form of a layer of one or more sheets or the like, such as a lawyer of two sheets.

The average thickness the sample collection pad (201, 101) is preferably less than 2 mm, such as in the range of 1 to 0.80 mm, preferably less than 1 mm, such as less than 0.95 mm, for example less than 0.85 mm, such as in the range of 0.85 to 0.80 mm, such as 0.83 mm. In one embodiment, the sample collection pad (201, 101) is in the form of a layer of two sheets, wherein the thickness of each sheet is less than 0.50 mm such as in the range of 0.49 to 0.40 mm.

In order to prevent or reduce any bias between the absorption of the one or more test analytes or any bias in the release of the one or more test analytes from the sample collection pad (201, 101), the sample collection pad (201, 101) may be pre-treated with a blocking buffer. In one embodiment, the blocking buffer is a PBS buffer comprising 1% BSA or a buffer comprising 10 mM Borate, 3% BSA, 1% PVP-40 and 0.25% Triton X100 pH 8.0.

In a particular embodiment, the sample collection pad (201, 101) is in the form of a cellulose material or a cellulose derivative such as nitrocellulose pre-treated with a blocking buffer, wherein the sample collection pad (201, 101) has a thickness in the range of 0.85 to 0.80 mm, such as 0.83 mm.

In a preferred embodiment of the present invention, the supporting member (202) of the separate swab (200) comprises an incision (206) on or near the edge of the distal end (204) of said supporting member (202). The lateral flow device (300) comprises a bulge (303) that fits with the incision (206) on the supporting member (202) and orientates and positions the distal end (204) of said supporting member (202) when the swab is inserted in the lateral flow device (300). Thus, the bulge/incision configuration secures that the swab and in particular the sample collection pad (201, 101) is orientated and positioned correctly in the lateral flow device (300). Thus, the bulge/incision configuration ensures that the swab and in particular the sample collection pad (201, 101) can only be inserted in the lateral flow device (300) such that the the sample collection pad (201, 101) of the swab (200) forms part of the porous support assembly (100), i.e. the sample collection pad is in contact with the other elements of the assembly. Accordingly, in one embodiment, the separate swab (200) comprises an incision (206) on or near the edge of the distal end (204) of said supporting member (202) and the lateral flow device (300) comprises a bulge (303) that fits with the incision (206) on the supporting member such that when inserted in the lateral flow device (300), the sample collection pad (201, 101) of the swab (200) forms part of the porous support assembly (100), i.e. the sample collection pad is in contact with the other elements of the assembly.

In another embodiment of the present invention, the lateral flow device (300) comprises an opening (304) configured to align with the aperture (205) of the swab such the sample collection pad (201, 101) is exposed through said opening (304), when the swab is inserted in the lateral flow device. Although the sample collection pad (201, 101) may be pre-wetted with running buffer, the opening allows the addition of (further) running buffer, where the separate swab (100) is in an inserted state in the lateral flow device (300).

When used in combination with the bulge/incision configuration, the sample collection pad (201, 101) is aligned correctly relative to the opening such that the running buffer is added to the sample collection pad (201, 101).

In one embodiment, the lateral flow device comprises a sample pad slot (306) configured to accept the distal end of said swab such that the position of the sample collection pad (201, 101, 301) in the lateral flow device is secured.

In another embodiment, the lateral flow device comprises a holding member (302) configured to hold the distal end of said swab and secure the position of the distal end of said swab comprising said sample collection pad (201, 101). In a further embodiment, the holding member is attached to the body of the lateral flow device by a hinge. In the open state, the distal end (204) of said supporting member (202) comprising the sample collection pad (201, 101, 301) may be inserted in the device, the insertion may be further facilitated by the presence of a sample pad slot (306) configured to accept the distal end of said swab. In the close position, the holding member is closed around the sample collection pad (201, 101, 301), which holds the sample collection pad (201, 101, 301) firmly in the lateral flow device (300). In one embodiment, the holding member (302) is configured to be closed down over the distal end of said swab and locked to the body of the lateral flow device. Preferably, the holding member (302) configuration of the device is used in combination with the bulge/incision configuration secures that the swab and in particular the sample collection pad (201, 101) is orientated and positioned correctly in the lateral flow device (300).

In a preferred embodiment, the holding member (302) comprises the opening (304) configured to align with the aperture (205) of the swab such the sample collection pad (201, 101) is exposed through said opening (304), when the swab is inserted in the lateral flow device. The opening (304) may be in the form of port such as in the form of a conical port with the wide base facing upwards and the narrow base facing downwards. In this configuration, (further) running buffer may be added to the sample collection pad (201, 101) in order to facilitate the lateral flow in the porous support assembly (100).

In the context of the present invention the term lateral flow refers to a liquid flow in which the dissolved or dispersed component(s) of the liquid (including the test analytes) migrates laterally with the liquid through the porous support assembly (100, referred to as capillary bed or lateral flow strip) with the proviso that component(s) are not permanently entrapped or by other means excluded from migrating in the liquid. Assay relying on such lateral flow of are referred to as lateral flow assay. Where the porous support assembly is preferable made of a non-bibulous material, the components in the liquid will travel at an essential equal speed through the capillary bed. If the porous support assembly is made of a bibulous material, the migration of one of more of the component may be affected by the material. If the porous support assembly comprises or consist of a bibulous material, the material may be treated with a blocking agent, such as PBS buffer comprising BSA and/or Triton X-100, in order to change the properties of the porous support assembly such that the flow characteristics is identical or essentially identical that of a non-bibulous material.

The lateral flow assay is based on the porous support assembly (100)—a capillary bed (such as porous paper or sintered polymer)—having the capacity to transport fluid by action of capillary forces. The porous support assembly (100) is an assembly of porous support elements, which elements are in in fluid communication with each other when fluid (such as a running buffer) is applied to the assembly. One of the porous support elements of the porous support assembly (100) is the sample collection pad (101, 200), which become part of the porous support assembly (100) when the swab is in the inserted position in the lateral flow device. The porous support assembly (100) is also referred to as the lateral flow assay strip.

In one embodiment of the present invention, the lateral flow device is constructed so as to form a porous support assembly (100), when it is mated with the sample collection pad attached to said swab, wherein the lateral flow device (300) mated with the sample collection pad comprise an elution zone (101), a conjugate zone (102) and a detection area (DA).

The conjugation zone may be an integrated part of a larger porous element of the porous support assembly (100), such as a porous support strip (107). The conjugation zone may also be in the form of an element of the porous support assembly (100). In a preferred embodiment, the conjugation zone is in the form of a conjugate pad (102).

The sample collection pad (101, 200) functions as a sponge and holds the test sample. Once it is soaked, the test sample, containing one or more test analytes, will migrate from sample collection pad (101, 200) into the adjacent element of the porous support assembly (100). The interphase between the sample collection pad (101, 200) and the adjacent element of the porous support assembly is referred to as the elution zone. The adjacent element of the porous support assembly is typically a conjugate zone, preferably in the form of a conjugate pad (102). The conjugate zone/conjugate pad (102) typically contains one or more indicator affinity molecule(s), such as affinity molecules tagged with detection probe designed to bind to the one or more test analytes within the test sample. The test sample and one or more affinity molecules are mixed and the one or more affinity molecules having affinity for one or more test analytes within the test sample will bind to each other while migrating further to a detection area (DA) that may contain a detection zone (105), and may contain an indicator zone (106), both with one or more stripes, where another set of one or more affinity molecules have been immobilized. By the time the test sample mixed with the affinity molecule(s) from the conjugate pad reaches the detection area (DA), the one or more analytes in the test sample will have been bound to the affinity molecule(s) from the conjugate pad. This complex will then in turn be bound by the affinity molecule(s) on the stripe(s) in the detection zone (105). After a while, when more and more fluid has passed the detection zone, detection probes accumulate, and the stripe changes color. The detection probes may e.g. be gold or latex particles conjugated to the affinity molecule(s) to prepare affinity molecules tagged with detection probes. The detection area (DA) may also comprise an indicator zone (106) which can function as a control to verify that the lateral flow assay has been conducted properly. Such indicator zone (106) may also comprise one or more stripes with affinity molecules immobilized that only binds to the affinity molecule(s) tagged with detection probes from the conjugate pad, whereas the affinity molecule(s) in the detection zone (105) bind to the complex between the analyte(s) and the indicator affinity molecule(s), such as the affinity molecule(s) tagged with detection probes from the conjugate pad. After passing the detection area (DA) the fluid enters the wicking pad (104), which generally receives fluid that has migrated through the entire porous support assembly (100). Thus in one embodiment, the detection area (DA) comprise a detection zone (105) containing one or more affinity molecule(s) for selectively retaining one or more test analyte(s) and optionally an indicator zone (106) containing one or more affinity molecule(s) for selectively retaining one or more indicator affinity molecule(s).

The detection zone (105) may be located upstream or downstream of the indicator zone (106). The lines or stripes in the detector zone or indicator zone may be disposed in a direction that is substantially perpendicular to the flow of the test sample. In some embodiments the lines may be in a direction that is substantially parallel to the flow of the test sample. The lines or stripes in the detection zone (105) or indicator zone (106) does not need to be lines or stripes, and can also be other shapes, such as e.g. dots or patterns.

In one embodiment, the lateral flow device further comprises a wicking pad (104). The wicking pad is part of the porous support assembly (100) and may assist in promoting capillary action and fluid flow from the sample pad (101), conjugate pad (102) through the detection area (DA).

In another embodiment, the lateral flow device comprises a backing material (108) on the backside of said porous support assembly (100) facing away from the elution zone. The backing layer (108) is liquid-impermeable so that fluid flowing through porous support assembly (100) does not leak through the backing layer (108). Examples of suitable materials for the support include, but are not limited to, glass; polymeric materials, such as polystyrene, polypropylene, polyester, polybutadiene, polyvinylchloride, polyamide, polycarbonate, epoxides, methacrylates, and polymelamine.

The porous support assembly (100) is an assembly of two or more porous elements, for example one or more porous elements and the sample collection pad (201, 101), where the swab (200) comprising the sample collection pad is inserted in the lateral flow assay device (300). The elements are preferably in the form of membranes, such as sheet like membranes. The porous support assembly (100) may have a thickness equal to or less than 4 mm (such as less than 4, 3, 2, 1 mm), and a width and a length both greater than the thickness. In some embodiments the width and length of the porous support assembly (100) are both greater (e.g. 3, 4, 5, 6, 7, 8, 9, 10, 50 times greater or up to 4, 5, 6, 7, 8, 9, 10, 50 times greater) than the thickness. In some embodiments the porous support assembly (100) is a square, such as a rectangle, and in some embodiments the porous support assembly (100) is circular. If the porous support assembly (100) is an irregular shape, i.e. different from a square or rectangle, then the width, length and thickness refers to the maximum values for such an irregular shape. For example the width of a circle will be the diameter. Examples of widths and lengths may be 4, 5, 6, 7, 8, 9, 10, 11, 12, 13, 14, 15, 16, 17, 18, 19, 20, 21, 22, 23, 24, 25, 26, 27, 28, 29, 30, 35, 40 mm, such as e.g. range of widths and lengths from 5-30 mm.

Thus in one embodiment, the porous support assembly (100) of the lateral flow device (300) has an average thickness equal to 4 mm or less, and a width and a length, both greater than the thickness, wherein the lateral flow device is configured to have a lateral flow direction (L) in the direction of a plane created by the width and the length of the porous support assembly.

In one embodiment, the lateral flow device comprises a reaction window (305) configured for visual inspection of the detection area (DA).

The diagnostic kit of the present invention may be used for testing analytes present on the skin and obtainable using the separate swab (200). In one embodiment, the one or more test analyte(s) are selected from the list consisting of: chemokines, interleukins, growth factors, hormones, enzymes, and other molecules present on the skin of a mammal, such as selected from the list consisting of: IL-1a, IL-1b, IL-1RA, IL-8, CCL-2, CCL-5, CCL-27, CXCL-1, CXCL-2, CXCL-9, Trappin2/Elafin, hBD-1, hBD-2, VEGF, and TSLP. In a preferred embodiment the test analytes are the combination of IL-8, IL-1a and IL-1RA.

As mentioned, the diagnostic kit of the present invention may be employed in point-of-care devices to detect the presence or absence of one or more test analytes within a test sample obtained from the skin using the separate swab (200) of the diagnostic kit. The readout may be done visually, i.e. presence or absence of a one or more coloured test lines also referred to as test stripes in a detection zone (105), and the confirmation/validation of the test may be done by the presence and/or absence of one or more coloured indicator lines/stripes in an indicator zone (106). The test may be qualitative (presence or absence) as well as quantitative, and the detection/quantification may be aided by reading equipment, or can be purely visual detection by the eye of the user of the lateral flow assay.

One aspect of the present invention provides a method for detecting the presence or quantity of one or more test analytes, the method comprising the following steps:
a) provide a separate swab (200) comprising a sample collection pad (201) as defined herein, wherein said sample pad comprises a test sample obtained from the a skin surface of a subject using said separate swab;
b) insert said swab comprising a sample collection pad containing said test sample in the lateral flow assay device adapted to receive said separate swab insert as defined in any of the preceding claims;
c) developing the lateral flow assay.

Another aspect of the present invention provides a method for detecting the presence or quantity of one or more test analytes, the method comprising the following steps:
a) collecting a test sample from the a skin surface of a subject using a separate swab (200) comprising a sample collection pad (201) as defined,
b) insert said swab comprising a sample collection pad containing said test sample in the lateral flow assay device adapted to receive said separate swab insert as defined in any of the preceding claims;
c) developing the lateral flow assay.

The subject is a mammal, preferably a human being. The test sample is obtained using the separate swab (200) comprising a sample collection pad (201), which is applied to the skin of the mammal, preferably the skin of a human being. The area of the skin may for example be the forehead, cheek, the inner arm or a part of the arm which is normally exposed to the sun. The separate swab may be applied to a pre-determined area, such an area not exceeding 5 cm2. The separate swab may also be applied in pre-determined time, such 5 seconds or 30 seconds. The test sample may also be collected by applying a pre-determined motion of the swab, such as a z-shaped motion of the swab on the skin.

The sampling may be assisted by wetting the sample collection pad (201) with a fixed volume of fluid. In a preferred embodiment, the sample collection pad (201) of the separate swab (200) is pre-wetted with a buffer before the sample collection, such as a with a fixed volume of a buffer. The buffer may be any suitable buffer such as a PBS buffer. The buffer used for pre-wetting the sample collection pad may be the same buffer used as running buffer in the lateral flow assay step of the procedure.

In another preferred embodiment, running buffer is added to sample collection pad inserted in the lateral flow assay device. The running buffer is added to the sample collection pad inserted in the lateral flow assay device to facilitate or provide sufficient fluid for the lateral flow in the porous support assembly (100) and the development of the assay. Where an opening (304) is present in the lateral flow device (300), the opening may be used as a port to add running buffer to the sample collection pad (201) inserted in the device.

In one embodiment, the lateral flow assay device comprises an elution zone (101) and a detection area (DA), and wherein said sample collection pad is the elution zone (101).

In another embodiment, the lateral flow device is constructed so as to form a porous support assembly (100), when it is mated with the sample collection pad attached to said swab, wherein the lateral flow device (300) mated with the sample collection pad comprise an elution zone (101), a conjugate zone (102) and a detection area (DA).

In one embodiment, the porous support assembly (100) has a thickness equal to 4 mm or less, and a width and a length, both greater than the thickness, wherein the lateral flow device is configured to have a lateral flow direction (L) substantially in the direction of a plane created by the width and the length of the porous support assembly (100).

In another embodiment, the detection area (DA) comprise a detection zone (105) containing one or more affinity molecule(s) for selectively retaining one or more test analyte(s) and optionally an indicator zone (106) containing one or more affinity molecule(s) for selectively retaining one or more indicator affinity molecule(s).

In general the present invention is directed to a diagnostic kit that provides an integrated system for detecting the presence or absence of one or more test analytes within a test sample obtained from the skin, over a broad range of possible concentrations of the one or more test analytes. In some embodiments the quantity of the one or more test analytes are also detected in a quantitative assay. The diagnostic kit employs a lateral flow assay device (300) and a separate swab (200, 301) and one or more assay reagents for detecting the one or more test analytes within the test sample. The assay reagents include affinity molecule(s) tagged with detection probes that are capable of producing a detection signal representing the presence or quantity of the one or more test analyte(s) in the test sample. One way of quantifying one or more of the test analyte(s) is by preparing suitable standard curves using known concentrations of the one or more test analyte(s).

The one or more test analyte(s) assayed using the method of the invention may be selected from the list consisting of: chemokines, interleukins, growth factors, hormones, enzymes, and other molecules present on the skin of a mammal, such as selected from the list consisting of: IL 1a, IL 1b, IL 1RA, IL 8, CCL 2, CCL 5, CCL 27, CXCL 1, CXCL 2, CXCL 9, Trappin2/Elafin, hBD 1, hBD 2, VEGF, and TSLP. In a preferred embodiment, the test analytes are the combination of IL-8, IL-1a and IL-1RA.

If desired, a suitable reading equipment, such as an optical reader may be used in some embodiments to measure the intensity of the probes. The actual configuration and structure of the optical reader may generally vary depending on the probes, which are to be measured. For example, optical detection techniques that may be utilized include, but are not limited to, luminescence (e.g. fluorescence, phosphorescence, etc.), absorbance (e.g. fluorescent or non-fluorescent), diffraction, and so on. Qualitative, quantitative, or semi-quantitative determination of the presence or concentration of an analyte may be achieved in accordance with the present invention. For instance, the amount of the analyte may be quantitatively or semi-quantitatively determined by using the intensities of the signals produced by detection probes bound at the detection zone (105) and the indicator zone (106).

In a preferred embodiment, an image of the detection area (DA) is captured using a suitable device for capturing images, such as a cell-phone comprising a camera. The image may subsequently be transmitted to a computer system (for example a remotely located server) comprising an image processor and a database, where the image is analysed, e.g. by extracting the image features and compare the features with corresponding features stored in a database. The computer system may then generate an output datum based on said image features, which may be transmitted to the user, e.g. back to the cell-phone used for capturing image.

Thus in one embodiment of the present invention, the method of the invention further comprises a step d) of capturing an image of the detection area (DA) and transmitting said image to a computer system comprising an image processor and a database, wherein the image features are extracted from the image by the image processor and said image features is stored in said database and, wherein said computer system generates a least one output datum based on said image features. In a further embodiment, the image is captured using a mobile device, such a cell phone configured to capture images. In yet a further embodiment, the output datum generated by the computer system is transferred to the mobile device.

When describing the embodiments of the present invention, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

The terms “comprising”, “comprise” and “comprises” herein are intended by the inventors to be optionally substitutable with the terms “consisting of”, “consist of” and “consists of”, respectively, in every instance.

The invention is further described in the following non-limiting items.

Item 1. A diagnostic kit for detecting the presence or quantity of one or more test analytes within a test sample taken from a skin surface of a mammal, the diagnostic kit comprising:

a) a separate swab (200, 301) configured to be used for collecting said test sample, wherein said swab comprising a sample collection pad (201, 101) attached to a supporting member (202),

b) a lateral flow assay device (300) configured to accept and hold said separate swab.

Item 2. The diagnostic kit of item 1 characterized in that the lateral flow assay device (300) comprises one or more porous elements and wherein the said sample collection pad (201, 101) is configured to form part of a porous support assembly (100) when the separate swab (200, 301) is inserted in said lateral flow assay device (300).

Item 3. The diagnostic kit of item 1 or 2, characterized in that said supporting member is configured with one proximal end (203) configured as a finger grip and opposite distal end (204) to which said sample collection pad (201) is attached.

Item 4. The diagnostic kit according to any one of items 1 to 3, characterized in that said is supporting member (202) is flexible along the longitudinal axis of supporting member.

Item 5. The diagnostic kit according to any one of items 1 to 4, characterized in that said distal (204) end of said supporting member (202) comprises an aperture (205) configured to be covered by the sample collection pad (201).

Item 6. The diagnostic kit according to any one of items 1 to 5, characterized in that said sample collection pad (201, 101) is attached to the supporting member (202) such that said sample collection pad covers said aperture (205).

Item 7. The diagnostic kit according to any one of items 1 to 6, characterized in that the area of the aperture (205) corresponds to at least 50% of the area of the sample collection pad (201, 101), such as at least 60% of the area of the sample collection pad, for example at least 70% of the sample collection pad, such as at least 70% of the sample collection pad, for example at least 80% of the sample collection pad, such as at least 90% of the sample collection pad, for example at least 95% of the sample collection pad.

Item 8. The diagnostic kit according to any one of items 1 to 7, characterized in that the sample collection pad (201, 101) is made of a cellulose material, a cellulose derivative such as nitrocellulose, polyether sulfone, polyethylene, nylon polyvinylidene fluoride (PVDF), polyester, polypropylene, glass fibers, cotton, or cloth.

Item 9. The diagnostic kit according to any one of items 1 to 8, characterized in that the sample collection pad (201, 101) is pre-treated with a blocking buffer, such as a PBS buffer comprising 1% BSA or a buffer comprising 10 mM Borate, 3% BSA, 1% PVP-40 and 0.25% Triton X100 pH 8.0.

Item 10. The diagnostic kit according to any one of items 1 to 9, characterized in that the sample collection pad (201, 101) is in the form of a sheet or the like.

Item 11. The diagnostic kit according to any one of items 1 to 10, characterized in that the sample collection pad (201, 101) is in the form of a layer of one or more sheets or the like, such as a lawyer of two sheets.

Item 12. The diagnostic kit according to any one of items 1 to 11, characterized in that the thickness of the sample collection pad (201, 101) is less than 2 mm, such as in the range of 1 to 0.80 mm, preferably less than 1 mm, such as less than 0.95 mm, for example less than 0.85 mm, such as in the range of 0.85 to 0.80 mm.

Item 13. The diagnostic kit according to any one of items 1 to 12, characterized in that the sample collection pad (201, 101) is in the form of a layer of two sheets, wherein the thickness of each sheet is less than 0.50 mm such as in the range of 0.49 to 0.40 mm.

Item 14. The diagnostic kit according to any one of items 1 to 13, characterized in that the sample collection pad (201, 101) is in the form of a cellulose material, a cellulose derivative such as nitrocellulose pre-treated with a blocking buffer, wherein the sample collection pad (201, 101) has a thickness in the range of 0.85 to 0.80 mm.

Item 15. The diagnostic kit according to any one of items 1 to 14, characterized in that the supporting member is made of a plastic material.

Item 16. The diagnostic kit according to any one of items 1 to 15, characterized in that the supporting member (202) is be made of a material that is flexible material such that the supporting member (202) will bend slightly when the sample collection pad (201, 101) is pressed against the skin and moved around on the skin to collect the test sample.

Item 17. The diagnostic kit according to any one of items 1 to 16, characterized in that the supporting member is made of a plastic material, where the thickness of the plastic material is less than about 2 mm, such as 1 mm or less, such as between 2 and 0.5 mm, such as between 2 and 1 mm Item 18. The diagnostic kit according to any one of items 1 to 17, characterized in that one edge of the said distal end (204) of said supporting member (202) comprises an incision (206) and wherein the lateral flow device comprises a bulge (303) configured to orientate and position the distal end of said supporting member when the swab is inserted in the lateral flow device.

Item 19. The diagnostic kit according to any one of items 1 to 18, characterized in that the lateral flow device comprises an opening (304) configured to align with the aperture (205) of the swab such the sample collection pad (201, 101) is exposed through said opening (304), when the swab is inserted in the lateral flow device.

Item 20. The diagnostic kit according to any one of items 1 to 19, characterized in that the lateral flow device comprises a sample pad slot (306) configured to accept the distal end of said swab such that the position of the sample collection pad (201, 101, 301) in the lateral flow device is secured.

Item 21. The diagnostic kit according to any one of items 1 to 20, characterized in that the lateral flow device comprises a holding member (302) configured to hold the distal end of said swab and secure the position of the distal end of said swab comprising said sample collection pad (201, 101).

Item 22. The diagnostic kit according to any one of items 1 to 21, characterized in that the holding member is attached to the body of the lateral flow device by a hinge.

Item 23. The diagnostic kit according to any one of items 1 to 22, characterized in that the holding member (302) is configured to be folded over the distal end of said swab and locked to the body of the lateral flow device.

Item 24. The diagnostic kit according to any one of items 1 to 23, characterized in that the holding member (302) comprises said opening (304).

Item 25. The diagnostic kit according to any one of items 1 to 24, characterized in that the said opening (304) is in the form of port such as in the form of a conical port with the wide base facing upwards and the narrow base facing downwards.

Item 26. The diagnostic kit according to any one of items 1 to 25, characterized in that the mammal is a human being.

Item 27. The diagnostic kit according to any one of items 1 to 26, characterized in that the lateral flow device is constructed so as to form a porous support assembly (100), when it is mated with the sample collection pad attached to said swab, wherein the lateral flow device (300) mated with the sample collection pad comprise an elution zone (101), a conjugate zone (102) and a detection area (DA).

Item 28. The diagnostic according to any one of items 1 to 27, characterized in that the conjugation zone is in the form of a conjugate pad (102).

Item 29. The diagnostic according to any one of items 1 to 28, characterized in that the lateral flow device further comprises a wicking pad (104).

Item 30. The diagnostic according to any one of items 1 to 29, characterized in that the lateral flow device comprises a backing material (108) on the backside of said porous support assembly (100) facing away from the elution zone.

Item 31. The diagnostic according to any one of items 1 to 30, characterized in that the porous support assembly has an average thickness equal to 4 mm or less, and a width and a length, both greater than the thickness, wherein the lateral flow device is configured to have a lateral flow direction (L) in the direction of a plane created by the width and the length of the porous support assembly (100).

Item 32. The diagnostic according to any one of items 1 to 31, characterized in that the detection area (DA) comprise a detection zone (105) containing one or more affinity molecule(s) for selectively retaining one or more test analyte(s) and optionally an indicator zone (106) containing one or more affinity molecule(s) for selectively retaining one or more indicator affinity molecule(s).

Item 33. The diagnostic according to any one of items 1 to 32, characterized in that the lateral flow device comprises a reaction window (305) configured for visual inspection of the detection area (DA).

Item 34. The diagnostic kit according to any one of items 1 to 33, characterized in that the one or more test analyte(s) are selected from the list consisting of: chemokines, interleukins, growth factors, hormones, enzymes, and other molecules present on the skin of a mammal, such as selected from the list consisting of: IL 1a, IL 1b, IL 1RA, IL 8, CCL 2, CCL 5, CCL 27, CXCL 1, CXCL 2, CXCL 9, Trappin2/Elafin, hBD 1, hBD 2, VEGF, and TSLP.

Item 35. The diagnostic kit according to any one of items 1 to 34, characterized in that the test analytes are IL-8, IL 1a and IL 1RA.

Item 36. The diagnostic kit according to any one of items 1 to 35, characterized in that it further comprises a separate container comprising a buffer suitable for prewetting the sample pad of said separate swab (200).

Item 37. The diagnostic kit according to item 36, characterized in that the separate container comprises running buffer.

Item 38. The diagnostic kit according to any one of items 1 to 37, characterized in that the separate swab (200) comprises an incision (206) on or near the edge of the distal end (204) of said supporting member (202) and the lateral flow device (300) comprises a bulge (303) that fits with the incision (206) on the supporting member such that when inserted in the lateral flow device (300), the sample collection pad (201, 101) of the swab (200) forms part of the porous support assembly (100).

Item 39. Method for detecting the presence or quantity of one or more test analytes, the method comprising the following steps:

a) provide a separate swab (200) comprising a sample collection pad (201) as defined in any of the preceding items, wherein said sample pad comprises a test sample obtained from the a skin surface of a mammal using said separate swab;

b) insert said swab comprising a sample collection pad containing said test sample in the lateral flow assay device adapted to receive said separate swab insert as defined in any of the preceding items,

c) developing the lateral flow assay device.

Item 40. The method according to item 39 further comprising adding running buffer to sample collection pad inserted the lateral flow assay device.

Item 41. The method to any one of items 39 to 40, wherein the lateral flow assay device comprises an elution zone (101) and a detection area (DA), and wherein said sample collection pad is the elution zone (101).

Item 42. The method according to any one of items 39 to 41, wherein the skin surface of the mammal is the skin of a human being.

Item 43. The method according to any one of items 39 to 42, wherein the lateral flow device is constructed so as to form a porous support assembly (100), when it is mated with the sample collection pad attached to said swab, wherein the lateral flow device (300) mated with the sample collection pad comprise an elution zone (101), a conjugate zone (102) and a detection area (DA).

Item 44. The method according to any one of items 39 to 43, wherein the porous support assembly (100) has a thickness equal to 4 mm or less, and a width and a length, both greater than the thickness, wherein the lateral flow device is configured to have a lateral flow direction (L) substantially in the direction of a plane created by the width and the length of the the porous support assembly (100).

Item 45. The method according to any one of items 39 to 44, wherein the detection area (DA) comprise a detection zone (105) containing one or more affinity molecule(s) for selectively retaining one or more test analyte(s) and optionally an indicator zone (106) containing one or more affinity molecule(s) for selectively retaining one or more indicator affinity molecule(s).

Item 46. The method according to any one of items 39 to 45, wherein said test analytes are IL-8, IL 1a and IL 1RA.

Item 47. The method according to any one of items 39 to 46 further comprising a step d) of capturing an image of the detection area (DA) and transmitting said image to a computer system comprising an image processor and a database, wherein the image features are extracted from the image by the image processor and said image features is stored in said database and, wherein said computer system generates a least one output datum based on said image features.

Item 48. The method according to item 47, wherein the image is captured using a mobile device, such a cell phone configured to capture images.

Item 49. The method according to item 48, wherein output datum generated by the computer system is transferred to the mobile device.

Item 50. The method according a to any one of items 39 to 49, wherein the sample collection pad (201, 101) is pre-treated with a blocking buffer, such as a PBS buffer comprising 1% BSA or a buffer comprising 10 mM Borate, 3% BSA, 1% PVP-40 and 0.25% Triton X100 pH 8.0.

Item 51. The method according a to any one of items 39 to 50, wherein the sample collection pad (201, 101) is prewetted prior to sample collection.

### EXAMPLES

Sample Pad Material and Treatment

### Example 1—In Vitro Testing of Sample Pad Materials

Different materials were tested as a sample collection material.

In vitro testing of the best material for sample collection and release was performed. 80 μl Standard protein solution (PBS containing 2 ng/ml IL8, 4 ng/ml IL1A and 8 ng/ml IL1RA recombinant proteins) was pipetted onto parafilm and adsorbed with 1×1 cm pieces of different possible sample pad material (blocked and unblocked). Sample pads were incubated 5 min at room temperature, inserted into SELF cassette, covered with FibroTx sample pad carrier (clear plastic strip, sample pad removed), cassette closed and 80 μl running buffer (PBS+1% Tween20) applied, results were read after 20 min run with Qiagen ESEQuant LR3 lateral flow reader, signal intensity results are shown in mV.

| | | IL8 | IL1A | IL1RA |
|---|---|---|---|---|
| | Material | (mV) | (mV) | (mV) |
| FibroTx | Cellulose, | 367.7 | 119.7 | 20.7 |
| sample pad | 0.95 mm | | | |
| C095 | Cellulose, | 448.1 | 99.1 | 0.0 |
| | 0.95 mm | | | |
| C095, blocked | Cellulose, | 162.0 | 89.4 | 51.8 |
| with 1% | 0.95 mm | | | |
| BSA/PBS | | | | |
| C083, | Cellulose, | 303.4 | 108.6 | 0.0 |
| blocked | 0.83 mm | | | |
| | Cellulose, | 337.7 | 151.2 | 78.4 |
| | 0.83 mm | | | |
| 2x C048 | Cellulose, | 266.6 | 81.1 | 0.0 |
| | 0.48 mm | | | |
| 2x C048, | Cellulose, | 330.7 | 167.3 | 112.9 |
| blocked | 0.48 mm | | | |
| 2x 111 | Glass | 278.2 | 151.5 | 47.2 |
| | Microfiber, | | | |
| | 0.28 mm | | | |
| 2x 111, | Glass | 297.5 | 132.3 | 92.0 |
| blocked | Microfiber, | | | |
| | 0.28 mm | | | |
| 2x 226 | Cotton | 337.1 | 105.5 | 0.0 |
| | 0.83 mm | | | |
| 2x 226, | Cotton | 220.3 | 125.3 | 41.8 |
| blocked | 0.83 mm | | | |
| 222 | Cotton, | 277.5 | 112.3 | 0.0 |
| | 0.83 mm | | | |
| 222 blocked | Cotton, | 327.9 | 126.9 | 85.2 |
| | 0.34 mm | | | |

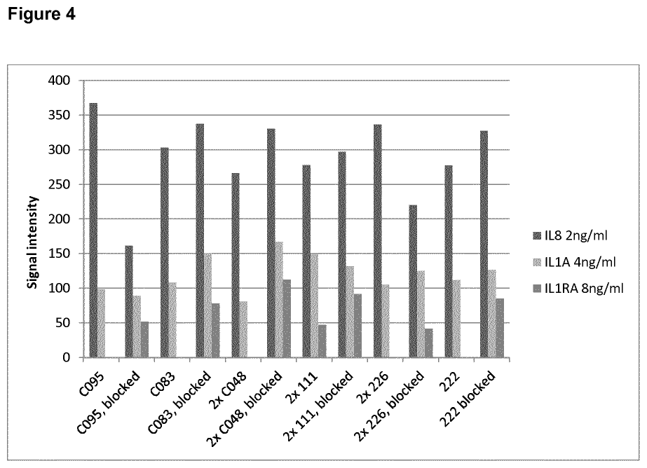

The data are presented in FIG. 4.

Unblocked sample pads resulted in lower signal intensities on IL1A test line compared to blocked sample pads of the same material and at the same time failed to release enough IL1RA to result in detectable test line for most sample pads tested. Blocked sample pads resulted in detectable IL1RA.

### Example 2—In Viva Testing of Sample Pad Materials

For in vivo testing best materials based on the in vitro results were selected: C083, C048 (2 layers), 111 (2 layers), 222, all blocked, were selected for testing on skin. First FibroTx sample pad (C095, unblocked) was also included.

Sample pads were placed on skin and covered with FibroTx sample pad bandage (sample pad was removed). 90 μl PBS was applied and sample pads were incubated on skin for 15 minutes. Sample pads were inserted into cassettes (including pad carriers), 80 μl running buffer (PBS+1% Tween20) applied, results were read after 20 min run with Qiagen ESEQuant LR3 lateral flow reader, signal intensity results are performed in mV. Skin from sun-exposed area (forehead) and sun-nonexposed area (inner side of a forearm) were tested in parallel.

| | IL8 | IL1A | IL1RA |
|---|---|---|---|
| Sample pad | (mV) | (mV) | (mV) |
| Area tested: | Forehead | | |
| FibroTx pad | 0 | 32.4 | 68.1 |
| | 0 | 14.8 | 46.3 |
| C083, blocked | 0 | 56.7 | 349.0 |
| 2x C048, blocked | 0 | 29.4 | 274.1 |
| 2x 111, blocked | 0 | 60.9 | 324.9 |
| 222 blocked | 0 | 25.9 | 196.9 |
| Area tested: | Inner arm | | |
| FibroTx pad | 0 | 30.5 | 0 |
| C083, blocked | 0 | 167.0 | 70.8 |
| 2x C048, blocked | 0 | 73.0 | 32.1 |
| 2x 111, blocked | 0 | 69.5 | 17.7 |
| 222 blocked | 0 | 59.9 | 18.2 |

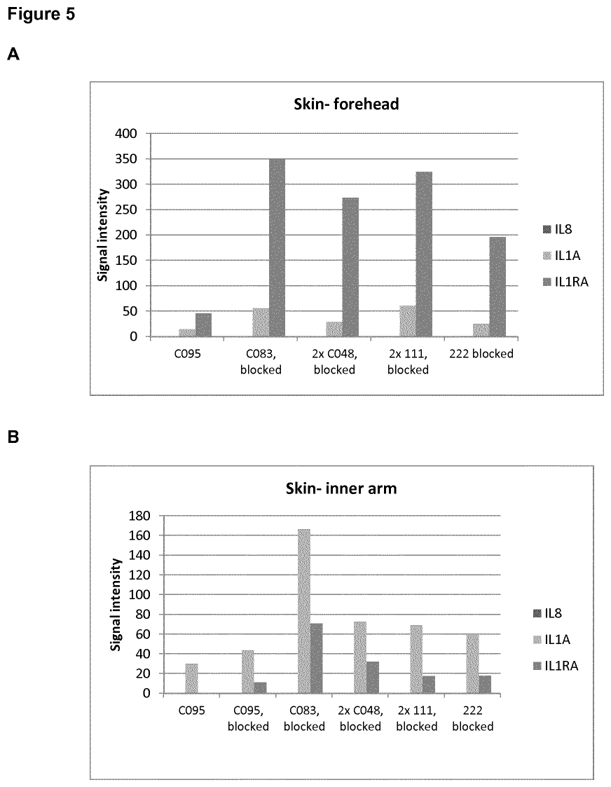

The data are presented in FIG. 5A (Forehead) and FIG. 5B (Inner arm)

On skin, C083 gave strongest signals, both from forehead and inner arm skin therefore this material was selected as a sample collection pad for FibroTx SELF.

### Example 3—In Vivo Testing Block Buffers

For this material 2 different blocking buffers were tested:
Simpler solution: 1% BSA+PBS
More complex solution: 10 mM Borate, 3% BSA, 1% PVP-40, 0.25% Triton-X100, pH8.0

Blocked sample pads (C083) were tested on inner side of a forearm skin and compared to unblocked sample pad.

Sample pads were placed on skin and covered with FibroTx sample pad bandage (sample pad was removed). 90 μl PBS was applied and sample pads were incubated on skin for 15 minutes. Sample pads were inserted into cassettes (including pad carriers), 80 μl running buffer (PBS+1% Tween20) applied, results were read after 20 min run with Qiagen ESEQuant LR3 lateral flow reader, signal intensity results are shown in mV.

| | IL8 | IL1A | |
|---|---|---|---|
| | (mV) | (mV) | IL1RA |
| C083 unblocked | 0 | 44.1 | 0 |
| C083 blocked with 1% BSA + PBS | 0 | 61.8 | 18.8 |
| C083 blocked with 10 mM Borate + | 0 | 104.9 | 27.4 |
| 3% BSA + 1% PVP-40 + 0.25% | | | |
| Triton-X100, pH8.0 | | | |

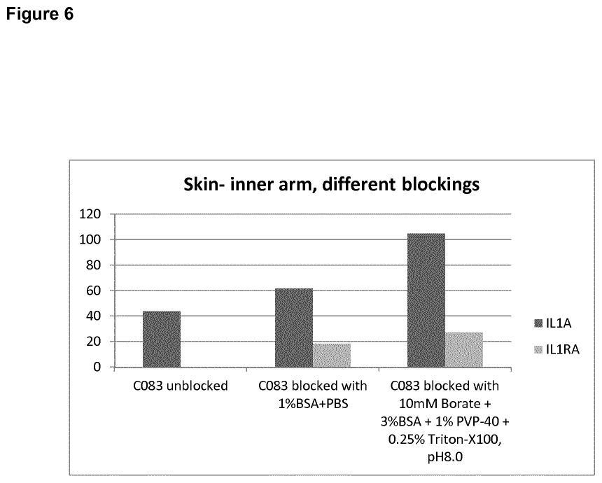

C083 sample pad blocked with more complex blocking buffer shows clearly best results for testing skin with FibroTx SELF. The data are presented in FIG. 6.

### Example 4—In Vivo Sample Collection Procedure

Biomarker sample can be obtained from skin by incubating the sample pad on skin (secured to skin with a bandage) or by swabbing (wiping/rubbing) the sample pad on the skin. These methods were compared to see if the swabbing could be used for simplifying the sample collection for the customer and to shorten the overall test time.

Sample collection by sample pad incubation on skin:

Sample pads (C083 blocked) were placed on skin and covered with FibroTx sample pad bandage. 2 drops of activation buffer (PBS) was applied and sample pads were hold on skin for 10 minutes.

Alternative sample collection method—rubbing the sample pad on skin:

Volunteers were given directions to add 2 drops of PBS onto the sample pad, remove the pad with the carrier from the bandage and rub the sample pad on skin, 2 different ways (not to touch the sample pad area from either side).
a) Wipe the sample collection area in a Z-shape motion
b) Wipe the sample collection area 5 seconds within approximately 5 cm area with circular motions.

After sample collection in either method described above, the sample pads were inserted into cassettes (including pad carriers), 2 drops of running buffer (PBS) applied, results were read after 20 min run with Qiagen ESEQuant LR3 lateral flow reader, signal intensity results are shown in mV.

| | Area | Sample | IL8 | IL1A | IL1RA |
|---|---|---|---|---|---|
| Volunteer | tested | collection | (mV) | (mV) | (mV) |
| JA | Forehead | Z-motion | 49.6 | 68.7 | 360.8 |
| | | 5″ rubbing | 29.4 | 70.8 | 277.0 |
| | | 10′ incub. | 0.0 | 43.5 | 320.3 |
| | Cheek | Z-motion | 16.1 | 40.5 | 186.0 |
| | | 5″ rubbing | 0.0 | 99.5 | 248.0 |
| | | 10′ incub. | 0.0 | 68.1 | 237.7 |
| AL | Forehead | Z-motion | 0.0 | 0.0 | 138.7 |
| | | 5″ rubbing | 11.6 | 49.1 | 346.5 |
| | | 10′ incub. | 0.0 | 30.7 | 104.0 |
| | Cheek | Z-motion | 12.1 | 12.1 | 106.4 |
| | | 5″ rubbing | 0.0 | 30.0 | 252.2 |
| | | 10′ incub. | 0.0 | 84.4 | 249.9 |
| AS | Forehead | Z-motion | 0.0 | 0.0 | 92.8 |
| | | 5″ rubbing | 0.0 | 32.1 | 225.8 |
| | | 10′ incub. | 0.0 | 37.4 | 298.8 |
| | Cheek | Z-motion | 0.0 | 25.0 | 72.4 |
| | | 5″ rubbing | 0.0 | 20.1 | 93.3 |
| | | 10′ incub. | 0.0 | 91.9 | 122.0 |

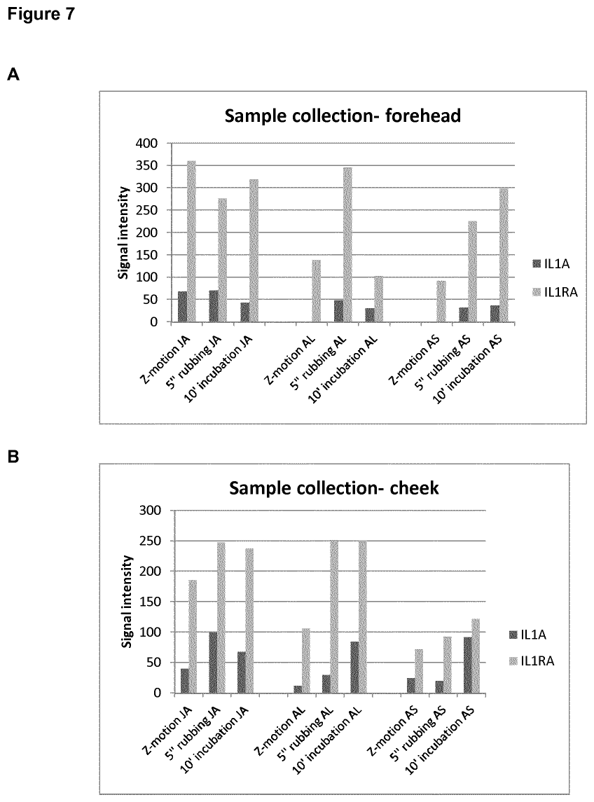

The data are presented in FIG. 7A (forehead) and FIG. 7B (cheek).

Swabbing the skin in Z-motion could obtain too little amount of sample (forehead).

For cosmetic purposes sample collection by swabbing was chosen as it considerably shortens and simplifies sample collection for the customer.

Longer swabbing time was tested also.

2 drops of PBS was added to sample pad and swabbed on skin in 3 different ways:
Wipe the sample collection area in a Z-shape motion
Wipe the sample collection area 5 seconds within approximately 5 cm area with circular motions.
Wipe the sample collection area 30 seconds within approximately 5 cm area with circular motions.

After sample collection the sample pads were centrifuged to eluate the PBS containing the collected sample, biomarker levels were determined using Enzyme-Linked ImmunoSorbent Assay, biomarker levels shown in ng/ml.

| | Sample | IL1A | IL1RA |
|---|---|---|---|
| Volunteer | collection | (ng/ml) | (ng/ml) |
| JA | Z-motion | 0.11 | 1.28 |
| | 5″ swabbing | 0.22 | 3.79 |
| | 30″ | 0.53 | 4.46 |
| | swabbing | | |
| AL | Z-motion | 0.03 | 2.17 |
| | 5″ swabbing | 0.09 | 5.07 |
| | 30″ | 0.38 | 6.60 |
| | swabbing | | |

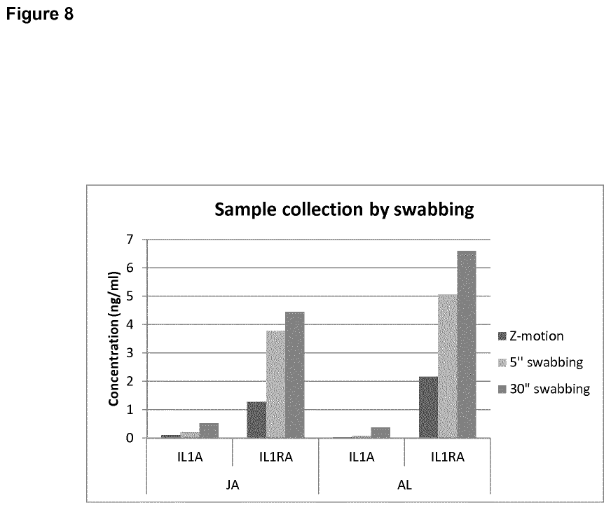

The data are presented in FIG. 8.

5″ swabbing obtains 2 times more material from skin compared to Z-motional swabbing. Longer swabbing time (30″) does increase the amount of obtained material even more, but swabbing for over 10″ causes crumbling of the sample pad material (depending on the intensity and pressure of swabbing by the customer) and therefore could not be suggested for functional tests.

### Example 5—In Vivo Testing of Effect of Skin Treatment Before Testing

The effect of skin treatment/washing prior to SELF testing was analysed to determine time needed before applying SELF bandage on skin after such treatments.

Inner arm was wiped 10× with cotton pad wetted with mQ water/ethanol or with cosmetic wipe.

Each treatment had control/untreated samples next to it, average of 3 controls was taken.

Sample pads (C083 blocked) were placed on skin and covered with FibroTx sample pad bandage at various times after this treatment. 80 μl of activation buffer (PBS) were applied and sample pads were incubated on skin for 15 minutes. Sample pads were inserted into cassettes, 80 μl running buffer (PBS) applied, results were read after 2 min run with Qiagen ESEQuant LR3 lateral flow reader, signal intensity results are shown in mV.

| | | | |
|---|---|---|---|
| Time after | IL8 | IL1A | IL1RA |
| treatment | (mV) | (mV) | (mV) |

Wiped with water

| | | | |
|---|---|---|---|
| 0 min | 0 | 122.2 | 49.5 |
| 5 min | 0 | 185.4 | 42.5 |
| 10 min | 0 | 155.4 | 45.8 |
| 15 min | 0 | 166.7 | 23.3 |
| 30 min | 0 | 250.7 | 59.9 |

Wiped with 70% EtOH

| | | | |
|---|---|---|---|
| 0 min | 0 | 123.8 | 32.6 |
| 5 min | 0 | 195.7 | 0 |
| 10 min | 0 | 196.6 | 16.2 |
| 15 min | 0 | 177.4 | 0 |

Wiped with cosmetic wipe

| 0 min | 0 | 173.8 | 34.1 |
|---|---|---|---|
| 5 min | 0 | 237 | 42.4 |
| 10 min | 0 | 195.1 | 53.7 |
| 15 min | 0 | 261.1 | 27.2 |
| | IL8 | IL1A | IL1RA |
| Control | (mV) | (mV) | (mV) |
| Parallel 1 | 0 | 278.2 | 24.5 |
| Parallel 2 | 0 | 180.9 | 19.2 |
| Parallel 3 | 0 | 134.6 | 61.2 |
| Average | 0 | 197.9 | 34.96667 |
| StDev | 0 | 73.29386 | 22.87276 |
| CV % | | 37.03581 | 65.41305 |

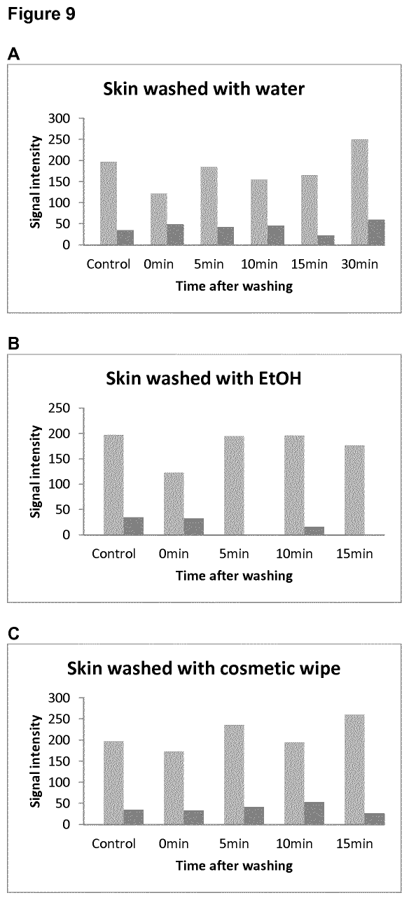

The data are presented in FIG. 9.
a) Washing with 70% ethanol seemed to have largest effects, mainly on IL1RA levels.
b) Right after treatments of IL1A and IL1RA had decreased slightly in all treatments (IL8 was not detected from healthy skin).
c) After 5 minutes, levels of IL1A and IL1RA seemed to have restored.
d) Before skin testing customer can follow his/her regular skin care routine: perform regular washing/cleaning and apply everyday cream/lotion/serum or such
e) Still it not advised to use extreme procedures to Your skin e.g heavy sunbathing, chemical/mechanical peels etc (unless it is required by the nature of the study) 3 days before the skin test and not wearing heavy/oily cream/serum/lotion or heavy make-up (e.g concealer, make-up cream, compact powder etc) during skin testing as it may affect the outcome of the result.

1. A kit for detecting the presence or quantity of one or more test analytes within a test sample obtained from a skin surface of a mammal, the kit comprising:
a) a separate swab configured to collect said test sample, wherein said swab comprises a sample collection pad attached to a supporting member wherein said supporting member comprises a proximal end configured for a finger grip and a distal end to which said sample collection pad is attached and, wherein the sample collection pad is in the form of a layer of one or more sheets,
b) a lateral flow assay device comprising one or more porous elements, wherein said lateral flow assay device is configured to accept and hold said separate swab,
wherein the said sample collection pad is configured to form part of a porous support assembly when the separate swab is inserted in said lateral flow assay device, and
wherein the lateral flow device connected with the sample collection pad comprise an elution zone, a conjugate zone and a detection area and, optionally a wicking pad, wherein the detection area comprises a detection zone containing one or more affinity molecule(s), which selectively retains one or more test analyte(s).

2-22. (canceled)

23. The kit according to claim 1, wherein one edge of the said distal end of said supporting member comprises an incision and wherein the lateral flow device comprises a bulge configured to orientate and position the distal end of said supporting member when the swab is inserted in the lateral flow device.

24. The kit according to claim 1, wherein the separate swab comprises an incision on the edge of the distal end of said supporting member and the lateral flow device comprises a bulge that fits with the incision on the supporting member such that when inserted in the lateral flow device, the sample collection pad of the swab forms part of the porous support assembly.

25. The kit according to claim 1, wherein said distal end of said supporting member comprises an aperture, and wherein said sample collection pad is attached to the supporting member such that said sample collection pad covers said aperture.

26. The kit according to claim 1, wherein the lateral flow device comprises an opening configured to align with the aperture of the swab such that the sample collection pad is exposed through said opening, when the swab is inserted in the lateral flow device.

27. The kit according to claim 1, wherein said supporting member is flexible along the longitudinal axis of supporting member.

28. The kit according to claim 1, wherein the supporting member is made of a material that is flexible such that the supporting member will bend slightly when the sample collection pad is pressed against the skin and moved around on the skin to collect the test sample.

29. The kit according to claim 1, wherein the supporting member is made of a plastic material and, wherein the thickness of the plastic material is less than about 2 mm.

30. The kit according to claim 1, wherein the sample collection pad is made of a cellulose material, or a cellulose derivative, polyether sulfone, polyethylene, nylon polyvinylidene fluoride (PVDF), polyester, polypropylene, glass fibers, cotton, or cloth and optionally that the sample collection pad is pre-treated with a blocking buffer.

31. The kit according to claim 1, wherein the sample collection pad is pre-treated with a blocking buffer.

32. The kit according to claim 1, wherein the sample collection pad is in the form of a layer of two sheets.

33. The kit according to claim 1, wherein the thickness of the sample collection pad is less than 2 mm.

34. The kit according to claim 1, wherein the sample collection pad is in the form of a layer of two sheets, and wherein the thickness of each sheet is less than 0.50 mm.

35. The kit according to claim 1, wherein the lateral flow device comprises a sample pad slot configured to accept the distal end of said swab such that the position of the sample collection pad in the lateral flow device is secured.

36. The kit according to claim 1, wherein the lateral flow device comprises a holding member configured to hold the distal end of said swab and secure the position of the distal end of said swab comprising said sample collection pad, wherein the holding member is configured to be folded over the distal end of said swab and locked to the body of the lateral flow device and wherein said holding member comprises said opening.

37. The kit according to claim 1, wherein said porous support assembly comprises an indicator zone containing one or more affinity molecule(s) for selectively retaining one or more indicator affinity molecule(s) and, optionally a reaction window configured for visual inspection of the detection area.

38. A method for detecting the presence or quantity of one or more test analytes, the method comprising:
a) providing a separate swab comprising a sample collection pad as defined in claim 1, wherein said sample pad comprises a test sample obtained from a skin surface of a mammal using said separate swab;
b) inserting said separate swab comprising a sample collection pad containing said test sample in the lateral flow assay device configured to receive said separate swab insert as defined in claim 1, optionally adding a running buffer to said sample collection pad inserted the lateral flow assay device; and
c) developing the lateral flow assay.

39. The method according to claim 38, wherein the sample collection pad is pre-treated with a blocking buffer.

40. The method according to claim 38, wherein the sample collection pad is prewetted prior to sample collection.

41. The method according to claim 38, wherein said test analytes are IL-8, IL-1a or IL-1RA.

42. The method according to claim 38, further comprising capturing an image of the detection area and transmitting said image to a computer system comprising an image processor and a database, wherein image features are extracted from the image by the image processor and said image features are stored in said database and, wherein said computer system generates a least one output datum based on said image features, wherein the image is captured using a mobile device configured to capture images and output datum generated by the computer system is transferred to said mobile device.
